(19) United States
(12) Patent Application Publication
(10) Pub. No.: US 2012/0197104 A1
(43) Pub. Date: Aug. 2, 2012

(54) SYSTEM AND METHODS FOR AUTOMATIC PLACEMENT OF SPATIAL SUPRESSION REGIONS IN MRI AND MRSI

(75) Inventors: Stefan Posse, Albuquerque, NM (US); Andre Van Der Kouwe, Charlestown, MA (US); Kunxiu Gao, Boxborough, MA (US); Weili Zheng, Troy, MI (US)

(73) Assignee: STC.UNM, Albuquerque, NM (US)

(21) Appl. No.: 13/322,332

(22) PCT Filed: Oct. 24, 2010
(86) PCT No.: PCT/US10/53885

§ 371 Date: Apr. 10, 2012

Related U.S. Application Data
(60) Provisional application No. 61/290,491, filed on Dec. 28, 2009.
(60) Provisional application No. 61/254,683, filed on Oct. 24, 2009.

(51) Int. Cl. A61B 5/055

(52) U.S. Cl. 600/410

(57) ABSTRACT

A system and methods for imaging a patient organ. The system includes a MRI imaging apparatus communicating with a memory and processor. The method aligns the organ with a standardized organ, and includes a step of spatially normalizing the standardized organ to the patient organ. The method also provides optimized slices of the standardized organ and translates optimized slices of standardized organ into optimized slices of the patient organ. The method images the patient organ according to the optimized slices of the patient organ.

### FIELD OF THE INVENTION

The present invention relates generally to MRI and MRSI imaging and, more specifically, to a system that provides automated placement of spatial suppression regions in MRI and MRSI scanning of patients.

### BACKGROUND OF THE INVENTION

Spatial suppression around a region of interest in a patient is standard routine in MRI. In almost all applications the placement of suppression regions is manual, which is time consuming and patient to operator error and bias. Improvements in suppression region placement are highly desirable. Spatial suppression of peripheral lipid-containing regions in volumetric MR spectroscopic imaging (MRSI) of the human brain also requires manual placement of a large number of outer volume suppression (OVS) slices. Similar to MRI, this manual placement is time consuming, prone to operator error and patient to intra-patient and inter-patient variability. However, 3-dimensional short echo time MR spectroscopic imaging in the brain is currently not routinely feasible due to the complexity of placing a large number of outer volume suppression slices around the brain, which is currently only feasible manually by a highly skilled operator or using a time consuming iterative optimization method for the placement of spatial suppression slices.

Suppression of overwhelming lipid and water signals from peripheral regions around the brain is desirable in proton MRSI to prevent spectral contamination inside the volume of interest (VOI) due to the point spread function. Point resolved spectroscopy (PRESS) and stimulated echo acquisition mode (STEAM) volume selection can be used to achieve the suppression by means of pre-localization of a rectangular box inside the brain. Another popular technique for whole brain MRSI is lipid nulling with Short T1 Inversion Recovery (STIR). As an alternative, frequency selective suppression methods provide powerful suppression using echo de-phasing, for example MEGA, BASING or BISTRO.

OVS using spatial pre-saturation can be employed for MRSI studies on clinical scanners and recent method development has focused on improving the suppression efficiency by optimizing RF pulse design, gradient switching schemes and timing of OVS modules. The feasibility of ultra-short TE high-speed MRSI in human brain using slice-selective Proton-Echo-Planar-Spectroscopic-Imaging (PEPSI) with eight OVS slices positioned along the periphery of the brain is one alternative. Another alternative is T1- and B1-insensitive outer volume suppression methods that employ highly selective broadband RF pulses that minimize chemical shift displacement artifacts at high field.

However, each of these processes performs manual positioning of suppression slices, which introduces operator-dependence and possible inter-patient and intra-patient variability of the volume-of-interest selection. Semi-automatic placement of 8 OVS slices for multi-slice MRSI based on a user-defined octagon shaped VOI has been considered, which facilitates clinical usage. However, for measurements at short TE in lateral cortical regions it is still necessary to manually place OVS slices to maximize volume coverage.

Manual placement of OVS slices requires considerable skill and time to balance the needs of completely covering peripheral brain regions with a limited number of OVS slices (to constrain T1-related losses in suppression) while minimizing the loss of lateral cortical brain regions, taking into consideration the OVS slice transition bandwidth and chemical shift artifacts. Moreover, manually placing a large number of OVS slices to obtain larger VOI coverage for volumetric MRSI is even more challenging and becomes unmanageable as the number of OVS slices increases.

There is a need to improve the process of positioning optimal OVS slices, especially when tracking lesion volume changes and related metabolic changes in clinical studies. It is clear that there is a demand for a system and methods that improve the positioning process in MRI and MRSI imaging and which may overcome problems of the prior art to reduce errors, time, and reliance on skilled professionals in the imaging process. The present invention satisfies these various demands.

### SUMMARY OF THE INVENTION

The present invention overcomes the problems of the conventional art described above. In particular, the present invention overcomes problems associated with manual placement of a patient during MRI and MRSI scanning of the patient.

Disclosed is an atlas-based approach for automatic placement of OVS slices by transforming OVS slices, which are optimally positioned on an atlas head to corresponding OVS slices on a patient's head using an affine transformation matrix. Optimal positioning of the OVS slices in atlas space was obtained using iterative optimization. This atlas-based method was validated in a retrospective analysis with up to 16 OVS slices using MPRAGE scans. The method was implemented on a clinical 3T scanner with additional automatic positioning of the MRSI slab and tested in 5 healthy patients using 3D short TE Proton-Echo-Planar-Spectroscopic-Imaging (PEPSI) with 8 OVS slices. Metabolite maps obtained with manually and automatically placed OVS slices showed consistent volume-of-interest (VOI) selection, and comparable degree of lipid suppression and number of usable voxels. The atlas-based method is fast (3 minutes processing time) and suitable for reducing intra-patient and inter-patient variability of VOI selection in multi-site cross sectional and longitudinal clinical MRSI studies.

Embodiments of the present invention using automated methods can delineate small volumes within a particular organ, such as around a breast lesion or prostate or a brain tumor mass, but this methodology has not been applied to delineate an entire organ. An iterative optimization approach to automatically place up to sixteen OVS slices in peripheral regions, which were delineated by skull-stripping using the FMRIB software library (FSL) brain extraction tool (BET) and demonstrated feasibility of automated short echo time (TE) 3D MRSI in a patient's brain on a clinical 3T scanner is provided. The resultant metabolic maps and spectra of this automated placement method were comparable to those acquired from manually placed OVS slices by a skilled operator. These automatic methods are capable of accurate placement of OVS slices on a patient by patient basis.

An atlas-based approach which automatically positions both the MRSI slab and the corresponding optimal OVS slices for volumetric MRSI is disclosed. The MRSI slab is first placed in a standard human brain atlas (the MNI512_T1_{—}1 mm head) to delineate the VOI of specific clinical interest, and up to sixteen OVS slices are automatically and optimally positioned to suppress peripheral lipid signals for the VOI defined by this slab. Subsequently, both the MRSI slab and the VOS slices are transformed to their corresponding positions in patient space through an affine matrix determined by the registration procedure that uses the patient structural scan and the atlas brain.

Long term efforts in automatic prospective prescription of MRI slice positions in the brain have been directed towards ensuring that observed differences in structural images were not caused by inaccurate prescription of scans or head movements. The atlas-based method, which is now available on clinical scanners of the major manufacturers, has shown its usefulness in clinical applications. Here, the slices are first selected in a probabilistic atlas representing the population and then aligned to an online localizer based on the rigid body registration matrix between the low resolution localizer and the atlas. Unfortunately, the ridge body registration considers only rotation and translation, which is not applicable for OVS slice placement due to the need for very precise positioning in peripheral region of the brain taking into account scaling and geometrical variability between the atlas brain and the patient brain. Therefore, it is desirable to use the affine transformation or nonlinear deformation.

### BRIEF DESCRIPTION OF THE DRAWINGS

The preferred embodiments of the invention will be described in conjunction with the appended drawing provided to illustrate and not to the limit the invention, where like designations denote like elements, and in which:

FIGS. 1(a)-(d) shows a system for automatically computing the optimal position and orientation of 8-16 saturation slices of a patient according to an embodiment of the present invention;

FIG. 2 shows a method for automatically computing the optimal position and orientation of 8-16 saturation slices of the patient according to an embodiment of the present invention;

FIG. 3(a) shows parameterization of suppression slice position;

FIG. 3(b) shows automatic positioning of sat bands in a sagittal view (left), and axial view (middle) and at the upper edge (right) of the 15 mm PEPSI slice;

FIG. 3(c) shows manual placement of sat bands by a highly trained operator;

FIG. 3(d) shows metabolite maps of residual lipids, NAA, Cho, Cr, Glu, and Ins (from left to right);

FIG. 3(e) shows the corresponding maps obtained with manual sat band placement (TE 15 ms, TR 2s; 1 cm³ voxel and 8.5 min scan time;

FIG. 4 shows matrices relating the target patient position with a scanned volume to an average atlas for rigid body transformations;

FIG. 5 is a flowchart showing reverse spatial mapping;

FIG. 6 shows automated cluster analysis of functional MR images after spatial normalization by mapping the Talairach Atlas into a patient's brain;

FIG. 7(a) shows a transformation of slice origin and angle of slice to suppress peripheral lipid regions around a patient brain;

FIG. 7(b) shows a selection of support points that define an origin and angles of a suppression slice in 3D;

FIG. 8(a)-(d) show placement of an MRSI slab and 16 OVS slices on an MNI template head and transformation to the subject head;

FIGS. 9(a)-(h) show an offline computation of 16 automatically positioned OVS slices in MNI space (top) and their transformed positions in subject space (bottom) in four different axial slice positions along the inferior to superior direction (a, z=80 mm; b, z=90 mm; c, z=110 mm; and d, z=120 mm);

FIGS. 10(a)-(f) show offline computation of the overlap of brain tissue with the OVS slices in cortical regions in axial (a), sagittal (b), and coronal (c) orientations in an individual's brain. (d) Cortical tissue covered by 16 OVS slices in axial view at the same axial slice level as in (a). Sagittal (e) and coronal (f) views of the intersection of the brain volume with the 80 mm MRSI slab (light+dark gray regions) and the 40 mm slab (light gray region);

FIGS. 11(a)-(g) show Metabolite maps obtained with automated placement of 8 OVS slices: (a) MRI, (b) Glu+Gln, (c) NAA+NAAG, (d) Cr+PCr, (e) tCho, (f) Ins, and (g) MM09

FIG. 12(a)-(f) show automated placement of 16 OVS slices in vivo shown on the scanner console in (a) sagittal orientation and at (b, c, d, e) different axial slice levels. The superior slice locations in (b) depicts 6 OVS slices of the upper ring and (c) depicts 7 OVS slices of the upper ring. The inner box is the shim region. The outer box delineates the field of view. The spectral grid in (d) shows the size of the encoded voxels and the localization of the 6 OVS slices from the upper OVS ring, which are outside of the head at this axial location. The inferior slice location in (e) depicts 7 of the 8 OVS slices that are part of the lower OVS ring. A representative spectrum from the scanner console with 0.38 cc acquired in 10:58 min is shown in (e). The only spectral processing that was applied was mild exponential filtering;

FIG. 13(a) shows Metabolite maps; and

FIGS. 13(b)-(c) show selected spectra from lateral and central regions in different slice locations and demonstrate comparably low levels of lipid contamination.

### DETAILED DESCRIPTION OF PREFERRED EMBODIMENTS

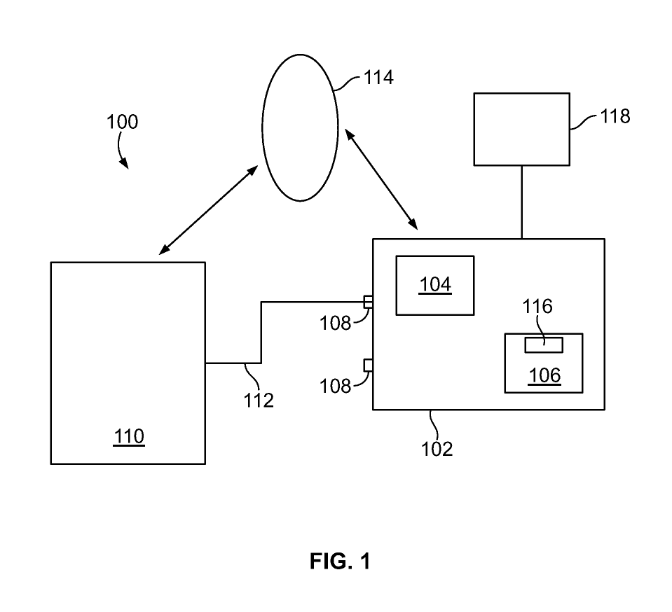

A system for optimizing placement of one or more suppression slices in peripheral regions in reference to a standardized space is shown in FIG. 1.

The system 100 of FIG. 1 may be a computer 102 that includes a processor 104 and data storage 106. The computer 102 further includes one or more data ports 108 configured for receiving patient data. The data may be transferred from a MRI or MRSI imaging apparatus 110 to the computer via a transmission line 112. Optionally, the computer 102 may be connected to the Internet 114 and receive patient data uploaded and sent from the imaging apparatus 110. The data storage 106 has an imaging module 116 stored thereon that includes a set of instructions causing the processor 104 to perform a series of steps when executing the instructions. The system 100 also includes a display 118 that displays scanned images output from processing the patient data which may assist a technician or provider with analyzing patient body parts including organs.

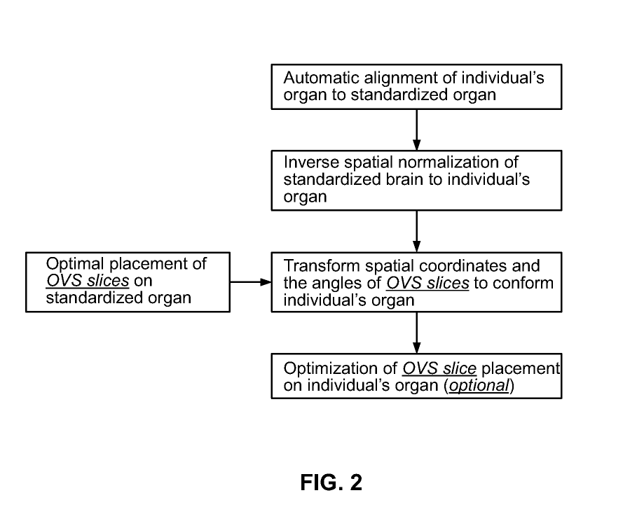

A preferred embodiment of the present invention includes the method described in FIG. 2. The method uses optimized placement of one or more suppression slices in peripheral regions in a reference to a standardized organ space (e.g. using an organ atlas). More specifically, the method may use a standardized brain space (e.g. using the MNI brain template), using manual or automated methods. The present method has automated alignment of the MR scanner's data acquisition coordinate system such that the image of a patient's organ (specifically the patient's brain) is co-registered to the standardized organ space (specifically the standardized brain space), which is already available from MR manufacturers (e.g. AutoAlign from Siemens Medical Solutions, Inc.). The present method has spatial normalization of the standardized organ (specifically the standardized brain) into the patient's organ (specifically the patient's brain) using an inverse of conventional spatial normalization that transforms a patient's space into a standardized space. The spatial resolution in this normalization step may be reduced from the original to accelerate the computation without significant loss of precision. The present method also has transformation of the spatial coordinates and the angles of the suppression region(s) from the standardized space into the patient's space using the spatial transformations described above, and optionally optimization of the suppression of peripheral regions around the organ by fine tuning the placement of the suppression region(s) using either segmented high-resolution MRI or chemical-shift selective MRI (e.g. lipid maps of the head using selective excitation or Dixon-type methods). This may be accomplished using optimization of slice positioning using an iterative algorithm based on a cost function.

The steps described above ensure very rapid and automated placement of suppression regions in MRI. The first step will be carried out only once for all applications. The advantages of the present invention as compared to conventional placement methods that use iterative optimization of suppression region placement are that the present method has: (i) reproducible placement within and across patients; (ii) spatial normalization that is fast (further optimization of suppression region placement, if required at all, is only minor); and (iii) a reliance on standard spatial transformation methods that are widely used and that are robust.

An exemplary embodiment of the present invention using the human brain in now described. The optimal placement of outer volume suppression slices on a standardized brain is determined. In a first step, the example embodiment recites computing the optimal position and orientation of 8-16 saturation slices to provide the best coverage of peripheral lipid containing regions around the brain. A high-resolution T1-weighted MP-RAGE scan of the standardized brain (e.g. MNI brain or Talairach brain) is segmented to obtain a mask for the lipid containing regions (e.g. using FSL). Optimal placement of the sat bands on the lipid containing regions involves optimization of the following cost function C=al1_{L}−blₒ−cl_{b}, where l_{L}, lₒ and l_{b}, are, respectively, the volumes of the intersections of the sat bands with peripheral lipid containing regions, with brain regions and the space around the head. The cost function reflects the balance between maximal lipid coverage, minimal loss of brain regions containing metabolite signals and reduction of sat band thickness outside of the head to minimize chemical shift artifacts. In order to compute the volumes of these intersections a convex hull described by the sat bands is computed. This algorithm is initiated by selecting the lowest MRI slice within the (thick) spectroscopic slab as a starting point. The maximization of the cost function is performed by iteratively computing the gradient of the cost with respect to the geometrical parameters of the sat bands, which are the azimuth angle φ, the elevation angle θ, the distances of the outer and inner surfaces of each sat band from the origin d1 and d2.

Since there is no analytical expression for the cost function, the gradient computation is performed by a numerical parametric approximation of first order. Since the algorithm is stable and converges, there is no justification for higher order approximations. Then, a 3-D version of the program applies the same gradient descent method to the entire lipid volume, gradually tilting the sat bands such that they follow the outer curvature of the brain. By using the output of the 2D program as the initial setting of the sat band parameters, a reasonable starting point for the 3D optimization is provided that assures the stability and convergence of the algorithm. This step is applied only once to prepare reference slice parameters for the following steps.

The methodology was applied in 3 patients with multiple scan replications in different sessions which were performed on a Siemens 4T scanner using a CP head coil. The sat band parameters were communicated to a Proton-Echo-Planar-Spectroscopic-Imaging (EPPSI) pulse sequence developed under Siemens Syngo-MR via text file. A maximum of 16 outer volume suppression slices can be defined.

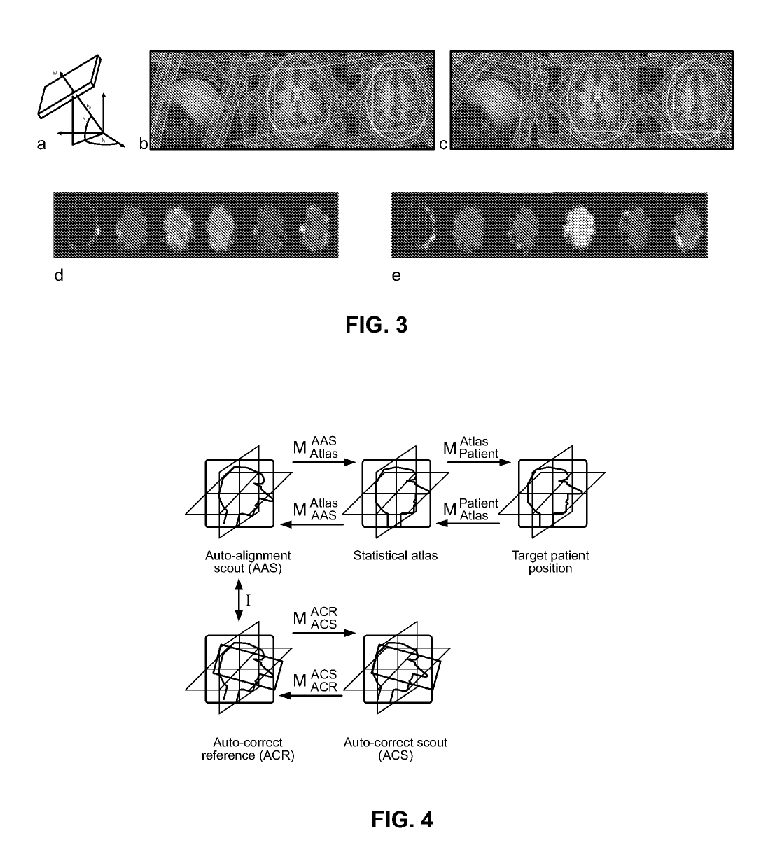

Upon loading, the sat bands are displayed on the graphics monitor overlaid on the high resolution MRI slices. Spectroscopic imaging data were collected with the PEPSI sequence using TR: 2 sec, TE: 15 msec, spatial matrix: 32×32, FOV: 240 mm, slice thickness: 15 mm, 8 averages and a total acquisition time of 8.5 minutes. A non-water suppressed reference scan was acquired for automatic phase and frequency shift correction as described previously. Data were reconstructed using even-odd echo separation as described previously. Spectroscopic images were computed based on LCModel fitting of 15 resonances. Spectral maps were thresholded at a CRLB of 50%. To compare the performance of the algorithm against a human operator, the following two metrics were used: (a) the integrated residual lipid signal divided by the integrated creatine signal across the entire spectroscopic slice and (b) the number of usable voxels with clearly identifiable metabolite signals. Automatic placement of sat bands was reproducible across different scanning sessions in the same patient. FIG. 3 shows automatically placed sat bands, which are similar to the placement by a highly trained human operator. Metabolite maps obtained from LCModel fitting using automatic band placement look very similar to those obtained with manual sat band placement, except for slight differences in peripheral gray matter. Table 1 shows the metrics defined above and Cramer-Rao lower bounds (CRLB) of different metabolites for automatic and manual sat band placement, confirming that the two method yield similar results. More specifically, Table 1 shows a Comparison of automated and manual placement in terms of # usable voxels inside the brain, integrated lipid signal in peripheral regions divided by integrated creatine signal in brain, and the minimum, maximum and average of the CRLB of selected metabolites in the PEPSI slice. Both methods provide similar results.

TABLE 1

| | # of | Integrated | | | | | |
|---|---|---|---|---|---|---|---|
| | usable | residual lipids/ | CRLB(NAA) | CRLB(Cho) | CRLB(Cr) | CRLB(Ins) | CRLB(Glu) |
| | voxels | integrated Cr | [%] | [%] | [%] | [%] | [%] |
| Automatic | 277 | 0.99 | Min: 2 | Min: 5 | Min: 3 | Min: 4 | Min: 5 |
| placement | | | Max: 46 | Max: 40 | Max: 49 | Max: 47 | Max: 46 |
| | | | Avg: 7.7 | Avg: 18 | Avg: 11 | Avg: 18 | Avg: 18 |
| Manual | 302 | 0.98 | Min: 2 | Min: 7 | Min: 4 | Min: 5 | Min: 3 |
| placement | | | Max: 49 | Max: 49 | Max: 40 | Max: 48 | Max: 48 |
| | | | Avg: 8.6 | Avg: 18 | Avg: 18 | Avg: 17 | Avg: 16 |

In clinical brain imaging protocols, the MR technician collects a quick localizer, and manually positions the subsequent scans using the localizer as guide. Autoalign presents a system for real-time on-line automatic positioning of slices using a statistical atlas, and for correcting the positioning between scans for subsequent patient movement as shown in FIG. 4. Accurate alignment ensures that left/right asymmetries reflect true patient anatomy, all patient scans are aligned in a consistent manner, and that patients returning for follow-up scans are positioned in precisely the same way so that images may be compared side-by-side to accurately monitor the progression of illness.

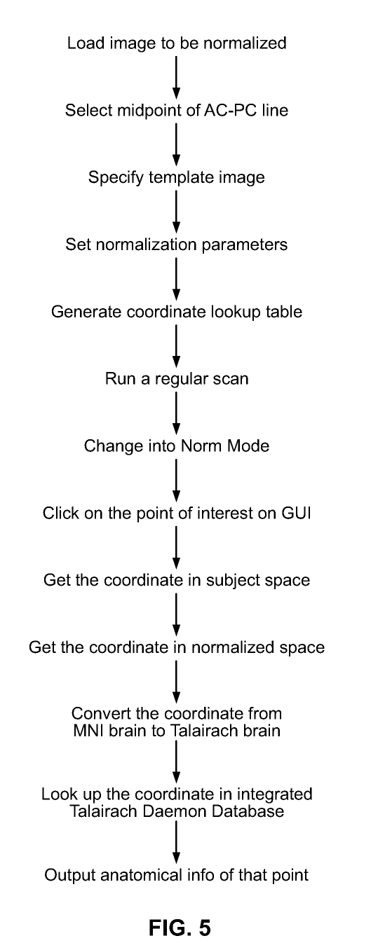

Inverse spatial normalization with automated referencing to the Talairach Brain Atlas is now considered. This approach maps a standardized brain (e.g. the MNI brain) onto a patient's brain. The processing steps of this reverse mapping are described in FIG. 5. The registration step can be directly adapted from SPM99 and generates a set of parameters which describe both linear affine transformation and nonlinear deformation of the reference image (which is the first image in the time series) to the template image (EPI template). Instead of the final resampling step of SPM to generate the normalized image, coordinates for each voxel in the normalized image (MNI space) are entered into a lookup table (S2N), and linked with the coordinates of the corresponding voxels in the reference image. Since spatially nonlinear transformations may expand a region during normalization several voxels in normalized space may be projected to the same voxel in patient space. In this case the corresponding source locations in normalized space are spatially averaged to generate a lookup table entry for the voxel in patient space. Alternatively, there might be locations in patient space that do not have any corresponding locations in normalized space in case a region is compressed during normalization. To solve this problem, the concept of “range searching” is used, i.e., if no corresponding normalized location is available, a pre-specified range is searched and the average of the closest coordinates is used. MNI coordinates are then transformed into Talairach space using Matthew Brett's formula and neuroanatomical information is then automatically assigned by querying the integrated Talairach Daemon database, which is also loaded in memory. The only manual user interaction needed is to select the midpoint of the AC-PC line on the reference scan to act as the origin of the coordinate system. Selectable options include the specification of the template image, the number of nonlinear iterations (12 by default), the degree of nonlinear regularization (medium by default), the bounding box and voxel resolution of the normalized image, and the brain mask image, consistent with the functionality of SPM99.

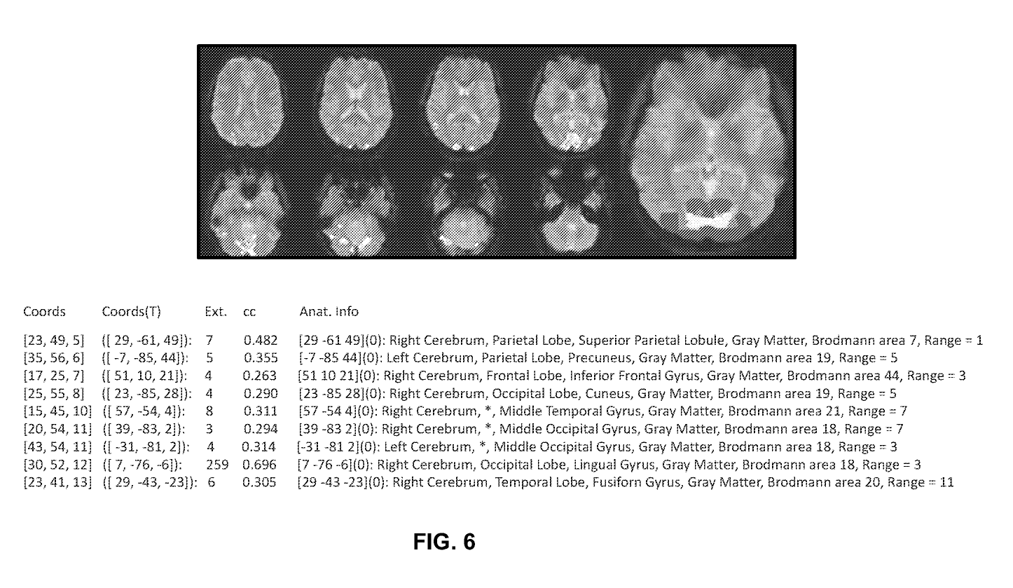

While generating the lookup table S2N using parameters from the registration step, an inverse lookup table (N2S) is also created. This allows mapping from normalized space to patient space so that neuroanatomically constrained regions using the Talairach Daemon database shown in FIG. 6 can be automatically selected. Neuroanatomical regions and combinations thereof can be selected from a list that is organized hierarchically: level 1 (Inter-Hemispheric, Left Cerebrum, Right Cerebrum, Left Cerebellum, Right Cerebellum, Left Brainstem, Right Brainstem), level 2 (lobes, medulla, pons, midbrain, sub-lobar, frontal-temporal space, 12 total), level 3 (gyri and related structures, 56 total), level 4 (cerebrospinal fluid, gray matter, white matter), level 5 (Brodmann areas and related structures, 73 regions total).

In order to validate spatial normalization, two highly trained raters evaluated independently the accuracy of automatic assignments generated by the proposed method by examining 23 gyri, sulci and other structures, for 7 different brains scanned with echo-planar-imaging (EPI) at 1.5 Tesla (TR: 2s, TE: 70 ms, FOV: 200 mm, 64×64 spatial matrix, 16 axial slices, 7 mm slice thickness). Results were rated on a scale from 0 to 4, corresponding to “Not Found”, “Disagree”, “Somewhat Disagree”, “Somewhat Agree” and “Agree”. Anatomical information was compared at three anatomical levels, i.e., left/right, lobe, gyrus/sulcus. The accuracy of spatial normalization was also examined using 12 clearly identifiable anatomical landmarks seen on 5 different brains scanned with EPI. The distance between these landmarks in images normalized with SPM99 and TurboFIRE was measured.

There was good agreement between rated anatomical structures and automatic anatomical assignments by TurboFIRE (Table 2). Agreement at lobular and at gyral/sulcal level was 94% and 92%, respectively. Supramarginal Gyrus, Uncus and Globus Pallidus are areas which were less reliably identified by the raters, mostly due to the limited image contrast in these regions in EPI data, due to their small size and due to low image resolution in EPI data. Distances between landmark coordinates measured with SPM99 and TurboFIRE ranged from 1 to 6 mm, with most of them (89%) being within 4 mm (see Table 3 below).

| | 0: Not Found; 1: Disagree; 2: Somewhat Disagree; 3: Somewhat Agree; 4: Agree | | | | | | | | | |
|---|---|---|---|---|---|---|---|---|---|---|
| | Lobular Level | | | | | Gyrus/Sulcus/Structure Level | | | | |
| | 0 | 1 | 2 | 3 | 4 | 0 | 1 | 2 | 3 | 4 |
| Frontal Lobe: | | | | | | | | | | |
| Superior Frontal Gyrus | | | | | 100.00% | | | | | 100.00% |
| Precentral Gyrus | | | | | 100.00% | | | | 3.57% | 92.86% |
| Middle Frontal Gyrus | | | | | 100.00% | | | | 3.57% | 96.43% |
| Medial Frontal Gyrus | | | | | 100.00% | | | | 3.57% | 96.43% |
| Inferior Frontal Gyrus | | | | | 100.00% | | | | | 100.00% |
| Anterior Cingulate Gyrus | | | | | 100.00% | | | | | 100.00% |
| Temporal Lobe: | | | | | | | | | | |
| Superior Temporal Gyrus | | | | | 100.00% | | | | | 100.00% |
| Middle Temporal Gyrus | | 7.14% | | | 92.86% | | | | | 100.00% |
| Inferior Temporal Gyrus | 7.14% | | | | 92.86% | 7.14% | | | 3.57% | 89.29% |
| Supramarginal Gyrus | | 17.86% | | | 82.14% | | 21.43% | | 7.14% | 71.43% |
| Parietal Lobe: | | | | | | | | | | |
| Postcentral Gyrus | | | | | 100.00% | | | | | 100.00% |
| Precuneus Gyrus | | 3.57% | | | 96.43% | | | | 3.57% | 96.43% |
| Superior Parietal Lobule | | | | | 100.00% | | | | 7.14% | 92.86% |
| Inferior Parietal Lobule | | | | | 100.00% | | | | | 100.00% |
| Occipital Lobe: | | | | | | | | | | |
| Middle Occipital Gyrus | 7.14% | | | | 92.86% | 7.14% | | | | 92.86% |
| Inferior Occipital Gyrus | 7.14% | | | | 92.86% | 7.14% | | | | 92.86% |
| Lingual Gyrus | | | | | 100.00% | | 3.57% | | 3.57% | 92.86% |
| Fusiform Gyrus | | 7.14% | | 3.57% | 89.29% | | | | 14.29% | 85.71% |
| Subcortical: | | | | | | | | | | |
| Caudate | | | | | 100.00% | | | 3.57% | 14.29% | 82.14% |
| Thalamus | 3.57% | 3.57% | | | 92.86% | 3.57% | | | | 96.43% |
| Globus Pallidus | 7.14% | 7.14% | | | 85.71% | 14.29% | | | | 85.71% |
| Parahippocampal Gyrus | | | | | 100.00% | | | | 3.57% | 96.43% |
| Uncus | 35.71% | 7.14% | | | 57.14% | 35.71% | | | | 64.29% |
| Average | 2.95% | 2.33% | 0.00% | 0.16% | 94.57% | 3.26% | 1.09% | 0.31% | 2.95% | 92.39% |
| Standard Deviation | 7.66% | 4.39% | 0.00% | 0.74% | 9.68% | 7.98% | 4.50% | 1.03% | 4.26% | 9.34% |

Table 2 above shows Validation of TurboFIRE spatial normalization by two expert raters: Their frequency of rating at lobular and gyral/sulcal level for 23 anatomical areas across the entire brain show good agreement of neuro-anatomical labels with observed neuro-anatomy.

Table 3 below shows validation of TurboFIRE spatial normalization by measuring the frequency of distances between landmark coordinates measured with SPM99 and TurboFIRE. The range of distances between 1 and 6 mm with mean distance of 3.8 mm is consistent with the voxel size of the EPI data.

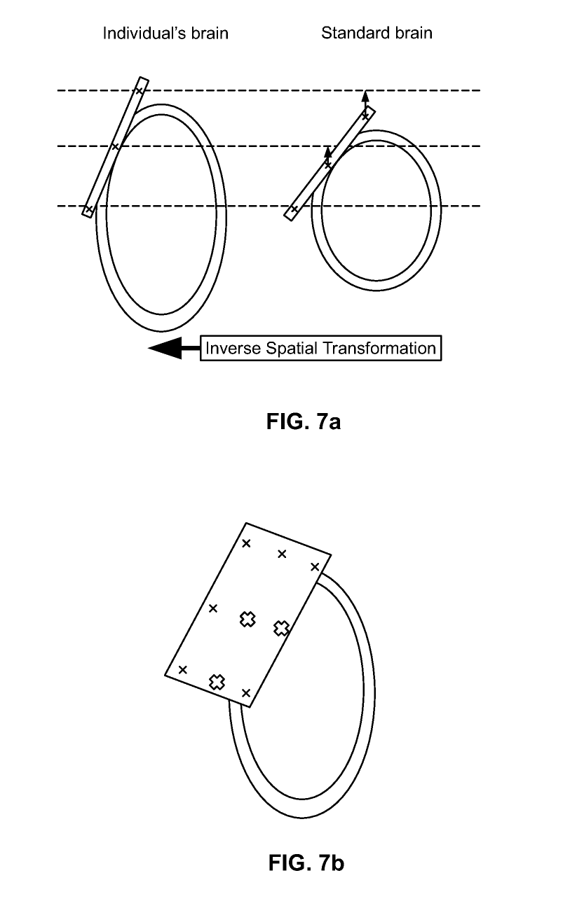

Transformation of spatial coordinates and angles of outer volume suppression slices to conform to patient's brain. A Method for positioning planar suppression slices is now disclosed. The objective is to perform a 6 parameter transformation (translation and rotation) for a planar slice. The location and angles of each suppression slice in standardized brain space are defined by selecting a minimum of three support points located within the planar slice as shown in FIG. 7. The first support point defines the origin of the slice; two support points at optimally selected distances along two orthogonal axes from the point of origin define the slice angle in reference to the standard brain. The coordinates of the three support points will be transformed as described in inverse spatial normalization with automated referencing to the Talairach Brain Atlas. The new support point coordinates define slice location and angles in reference to the patient's brain. More than 3 support points along more than 2 in-plane axes may be selected to achieve greater precision in position the suppression slice in reference to a patient's brain. In case more than support 3 points are selected it will be necessary to assign different weights to each point to take into consideration nonlinear local spatial distortions as a consequence of spatial normalization. Support points located closer to the slice origin will receive greater weights than support points located further away from the slice origin.

A second method for positioning planar suppression slices is now disclosed. The origin of the suppression slice transforms as described in the above method. The orientation of the saturation bands under scaling transformations (as part of spatial

Distance (mm)

| | 1 | 2 | 3 | 4 | 5 | 6 |
|---|---|---|---|---|---|---|
| % of Landmarks | 8 | 19 | 32 | 30 | 9 | 2 |

normalization) is computed as follows: The problem of how to orient each saturation band given an arbitrary scaling of the three axes, as implemented on the Siemens scanner, reduces to the question of how the normal vector to the slab changes under this scaling transformation. The in-plane rotation (about the slab normal vector) doesn't matter for saturation bands because they are parallel to the plane that is tangent to the surface of the head and are equally effective for any arbitrary orientation in this plane. Let K represent the scaling transformation:

$$K = [ \begin{matrix}{k}_{1} & 0 & 0 \\ 0 & {k}_{2} & 0 \\ 0 & 0 & {k}_{3}\end{matrix} ]$$

Let ₙ represent the slice normal vector in the original coordinate system and let _{n′} represent the slice normal vector in the transformed coordinate system. Then it can be shown that:

$${n}^{′} = [ \begin{matrix}{k}_{2} {k}_{3} & 0 & 0 \\ 0 & {k}_{1} {k}_{3} & 0 \\ 0 & 0 & {k}_{1} {k}_{2}\end{matrix} ] n$$

A method for positioning curved suppression slices has an objective to perform a spatially nonlinear transformation for a curved slice. The position of each suppression slice in standardized brain space is defined by multiple support points located within the curved slice. The density of point depends on the topology of the suppression slice and the range of spatial basis sets for the nonlinear transformation: it may be sparse in case of low dimensional curvature of the suppression slice or it may be very high in case of a slice that follows closely the shape of the lipid containing regions around the brain. The coordinates of these support points will be transformed as described in Inverse spatial normalization with automated referencing to the Talairach Brain Atlas. The new support point coordinates define the suppression slice in reference to the patient's brain. Weights may be used for these support points to constrain the curvature of the transformed suppression slice.

Another method for suppressing selected brain regions based on a standard brain atlas database has selection of brain regions defined in a brain atlas database, such as the Talairach Daemon, and enables 3D excitation and suppression of brain regions. This region selection may be accomplished using the methods described in Inverse spatial normalization with automated referencing to the Talairach Brain Atlas.

Optimization of the placement of the suppression regions may be accomplished by increasing or decreasing the dimension of the suppression regions perpendicular to the surface of the organ to match the outer edge of the desired suppression region (specifically, the peripheral lipid containing regions around the brain) in a patient's space. Alternatively, it is possible to employ the method described in optimal placement of outer volume suppression slices on a standardized brain.

Based on the framework of atlas-based slice prescription, a solution is provided to automatically place the OVS slices online in 3D MRSI. In a first step the MRSI slab is placed in atlas space to define a VOI of specific clinical interest. The thickness and orientation of the slab is chosen based on the number of available OVS slices, the expected magnetic field inhomogeneity within the VOI and TE. The largest variation and strongest local magnetic field inhomogeneity in previous studies were measured in inferior frontal brain areas. This area was therefore excluded in the slab placement. Long TE MRSI scans are more tolerant to lipid contamination and magnetic field inhomogeneity, enabling the use of larger VOIs. Short TE MRSI scans are less tolerant to magnetic field inhomogeneity, constraining the VOI size. The OVS slices are automatically placed using an optimization method to completely cover lipid containing peripheral areas. During the actual MRSI scan session, high resolution structural scans of the subject are used to compute the affine transformation matrix between the atlas brain and the subject brain. Then the prescriptions of the MRSI slab and the OVS slices are mapped to subject space using the inverse of the affine transformation.

### Placement of the MRSI Slab and OVS Slices in Atlas Space (Offline)

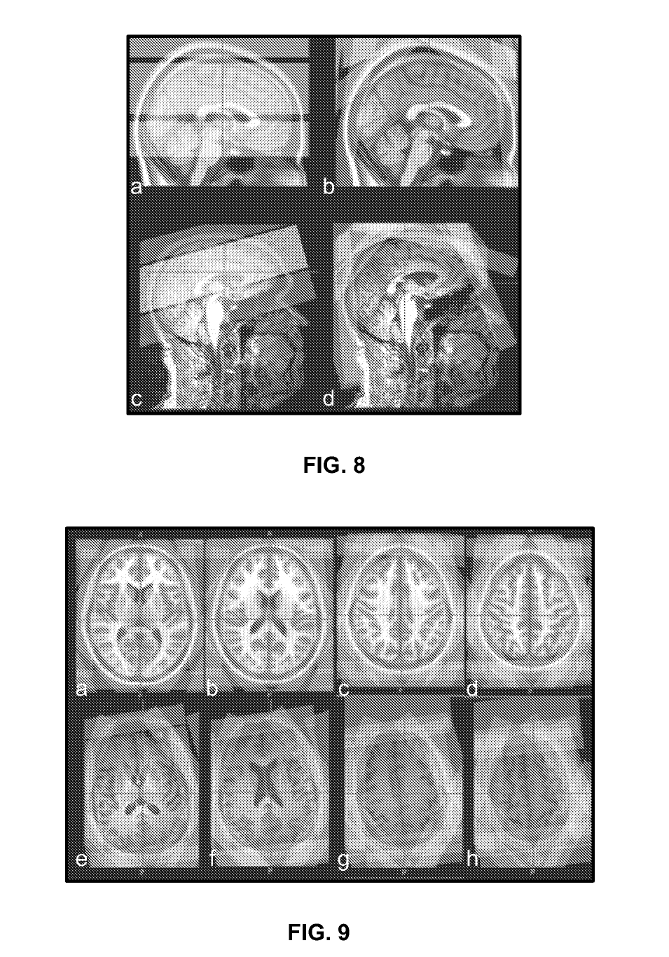

The first step is to manually place the MRSI slab in the brain atlas to define the VOI. For short TE 3D MRSI in the upper cerebrum a thick slab is positioned along a line through the superior surface of the anterior commissure and the center of the posterior commissure (AC/PC orientation) extending upwards from the middle of the ventricles to the top of the brain, which avoids frontal areas with large magnetic field inhomogeneity. The VOI is thus defined as the brain volume covered by the MRSI slab. This VOI may be changed according to the clinical interest, but shimming conditions in vivo need to be taken into consideration. In case of 16 OVS slices, two of the OVS slices were placed directly inferior and superior, in parallel to the MRSI slab in order to reduce edge artifact from imperfections of 3D RF excitation (FIG. 8), and the rest are placed automatically in two rings to form a convex hull. In the case of 8 OVS slices a single ring is used. First, the lipid-containing peripheral regions in a brain atlas are identified using the brain/non-brain segmentation tools BET. The performance of BET is satisfactory here due to the absence of intensity inhomogeneity and other artifacts in the template. Then the OVS slices are automatically placed using an iterative optimization method. Note that this offline optimization procedure is performed only once for all subsequent in vivo scanning.

### Registration of the Subject Head to the Atlas Head (Online)

The registration problem here is to find the set of parameters describing the transformation matrix from the source image in subject space to the reference image in atlas space, which maximizes the “similarity” between these two images in atlas space. The transformation function could be a linear function or a combination of some nonlinear basis functions. Here the FLIRT (FMRIB's Linear Image Registration Tool) is used, a widely used fully automated robust and accurate tool, which uses an intensity based cost function for linear (affine) intra-modal structural brain image registration. To further improve the precision of registration, additional factors such as the interpolation method, the intensity inhomogeneity of subject images due to B1-inhomogeneity, orientations of images, and the symmetry of registration were considered.

### Transformation of the Prescription of Slices to Subject Space (Online)

The affine transformation using FLIRT consists of translation, rotation, scaling and shear. The slice S in atlas space is defined by a normal vector N, which is represented by three orthogonal vectors (Nₓ,N_{y},N_{z}), and the slice center (Pₓ,P_{y},P_{z}) with respect to a generic 3D Cartesian coordinate system:

$$S = ⌊ \begin{matrix} & & & {P}_{x} \\ {N}_{x} & {N}_{y} & {N}_{z} & {P}_{y} \\ & & & {P}_{z} \\ 0 & 0 & 0 & 1\end{matrix} ⌋ \qquad ( 1 )$$

The affine transformation generates a slice S′ in subject space:

S′=Mᵣ⁻¹S (2)

where the transformed center is represented by (Pₓ′,P_{y}′,P_{z}′). However, the transformed vectors (Nₓ′,N_{y}′,N_{z}′) are no longer orthogonal if a shear transformation is involved. To account for this, three new orthogonal vectors (Nₓ″,N_{y}″,N_{z}″) are reconstructed as follows:

Nₓ″=Nₓ′

N_{z}″=Nₓ′×N_{y}′

N_{y}″=N_{z}′×Nₓ′ (3)

where x is the vector product operator, used here to orthogonalize vectors N_{y} and N_{z} with respect to N.
The transformed slice S′ is thus represented by

$${S}^{′} = ⌊ \begin{matrix} & & & {P}_{x}^{′} \\ {N}_{x}^{″} & {N}_{y }^{″} & {N}_{z}^{″} & {P}_{y}^{′} \\ & & & {P}_{z}^{′} \\ 0 & 0 & 0 & 1\end{matrix} ⌋ \qquad ( 4 )$$

In the implementation, the output OVS slice center and normal vectors were specified in accordance with the Siemens patient coordinate system LPS (Left-Posterior-Superior), which is used in the Siemens console GUI. The MNI template is in the Neurological (Right-Anterior-Superior=RAS) orientation and the FMRIB Software Library (FSL) uses the Radiological (Left-Anterior-Superior=LAS) coordinate system. To ensure internal consistency all intermediate images, transformations and prescription of slices conformed to RAS.

### Implementation of the OVS Placement Pipeline

The placement of the MRSI slab and the OVS slices in MNI space in Step 1 is carried out offline only once, while the subsequent steps are performed online for each subject brain during an in vivo MRSI experiment.

Step 1: The MNI template (MNI512_T1_{—}1 mm head [19]) was used in RAS orientation. The MRSI slab was manually placed on the MNI head and oriented in parallel with the AC-PC line (the anterior axis in the RAS coordinate system) extending in the superior direction (FIG. 8). The slab thickness was chosen to be either 40 mm for the 8 OVS slice implementation or 52 mm for the 16 OVS slice implementation. This selection of slab thickness in vivo was chosen to maximize brain volume coverage while minimizing loss of cortical tissue and line broadening due to magnetic field inhomogeneity in inferior frontal cortex. For offline simulation the slab thickness for 16 OVS slice was also 52 mm. The iterative optimization procedure for these OVS slices yielded a slice thickness in MNI space ranging from 19.2 mm to 22 mm. This thickness was manually extended to a uniform 25 mm, taking into consideration the finite transition width of the suppression slice profile and chemical shift displacement. The slice thickness of these two OVS slices was optimized manually to suppress signals from the nasal cavities (slice 1, inferior to the MRSI slab, thickness: 40 mm) and to fully cover superior lipid containing regions (slice 16, superior to the MRSI slab, thickness: 30 mm).

Step 2: High resolution T1-weighted structural scans acquired with the Siemens MPRAGE sequence were used to obtain the affine transformation matrix between the MNI brain and the subject brain. The MPRAGE scans in DICOM format were transferred to an external 64-bit Dell T7400 workstation and converted to a 3D volume in NIFTII format (.nii) using the FreeSurfer (version 3.0.4) (http://surfernmr.mgh.harvard.edu/) function mri_convert.

Step 3: It was necessary to adjust the image intensity profile to reduce adverse effects of intensity inhomogeneity on registration accuracy. The FreeSurfer module mri_nu_correct.mni was used to correct the image intensity inhomogeneity of the subject head 3D volume. It uses the Nonparametric Non-uniformity intensity Normalization method (N3) that does not require a tissue model, is independent of pulse sequence and is insensitive to pathology.

Step 4: The inhomogeneity-corrected 3D volume was re-sliced to RAS orientation with a 1×1×1 mm³ voxel size using a Matlab routine reslice_nii, available for download from http://www.rotman-baycrest.on.ca/˜jimmy/NIFTI/.

Step 5: The affine transformation matrix Mᵣ was determined by registering the subject head to the MNI head using the FSL function FLIRT. Here, Mᵣ provided by FLIRT was defined in the native image coordinate system (centered at one of the image volume corners) instead of the RAS system.

Step 6: A Matlab script was developed in-house to map the prescription (center and normal vector) of the MRSI slab and the OVS slices in MNI space to subject space using the inverse of the above affine matrix Mᵣ⁻¹

S′=MₛMᵣ⁻¹Mₐ⁻¹S (5)

where Mₛ and Mₐ are the voxel to RAS transformation matrices between the subject brain and the MNI brain.

Step 6: For visualization purposes using fslview, the transformed prescriptions of the MRSI slab and OVS slices were reconstructed to form 3D slabs in the subject space that were overlaid on the high-resolution MPRAGE scan. The MRSI slab and OVS slice prescriptions were written to an ASCII file, which was transferred to the scanner console and read by a modified PEPSI pulse sequence (see below). The placement of the slices was shown superimposed on the subject's localizer scan by the scanner GUI.

### Subjects and Data Acquisition

Data were collected on Siemens 3T TIM Trio scanners (Siemens Medical Solutions, Inc.) equipped with Avanto gradient system and 12 channel array head coil. High-resolution T₁-weighted MPRAGE (Magnetization Prepared RApid Gradient Echo) scans were acquired with TR: 1810 ms, TI: 900 ms, TE: 2.52 ms, flip angle: 8°, bandwidth: 651 Hz/Px, 160 or 192 sagittal slices with 256×256 in-plane resolution, and isotropic 1 mm voxel dimensions. High-resolution multi-slice T₂-weighted turbo spin-echo scans with the same slice orientation as the PEPSI scan were acquired for manual placement of the OVS slices. MRSI data acquisition was performed using the PEPSI pulse sequence, using a spectral width of 1087 Hz and a digital spectral resolution of 1 Hz. The GUI of the Siemens scanner allows manual placement of up to 8 OVS slices. For automated OVS placement the PEPSI sequence was modified to read the OVS slice offsets, rotation angles and thicknesses from an ASCII text file described above. The number of OVS modules was increased to 16, which required elongating the duration of the first two water suppression modules to maintain consistent timing of the water suppression modules. Gradient crusher orientations and amplitudes were carefully chosen to avoid secondary echoes. The GUI was modified to display a user selectable set of 8 of the 16 OVS slices overlaid on the T₂-weighted turbo spin-echo scans to assess OVS slice placement. The MRSI slab origin and orientation were entered manually from the ASCII text file described above.

3D PEPSI data for comparing manual and automated placement of 8 OVS slices was collected in 11 patients from a 40 mm thick slab in AC/PC orientation extending from the middle of the ventricles in the superior direction: TR: 2 s, TE: 15 ms, FOV: 226×226×55 mm, spatial matrix: 32×32×8 with elliptical sampling in the sagittal (y-z) plane, nominal voxel size: 0.34 cm³, scan time: 4:43 min. Manual placement of the MRSI slab and 8 OVS slices was performed by an experienced operator (SP). Water suppressed (WS) data were acquired with a single average using first- and second-order autoshimming and automated adjustment of water suppression. A non-water suppressed (NWS) reference scan with 1 signal average using a shorter TR (1 s) was also collected.

3D PEPSI data for automated placement of 16 OVS slices was collected in 3 subjects from a 52 mm thick slab in AC/PC orientation extending from the basal ganglia in superior direction: TR: 2s, TE: 20 ms, FOV: 226×226×60 mm, spatial matrix: 32×32×8, elliptical sampling in the sagittal (y-z) plane, nominal voxel size: 0.37 cm³, scan time: 4:43 min. In one subject a spatial matrix of 32×32×16 with FOV 226×226×120 mm and 10:58 min scan time was used. NWS reference scans were collected with single average using TR: 1 s.

### MRSI Data Reconstruction and Quantification

Reconstruction of PEPSI data was performed online using an ICE program that performs ramp sampling correction, removal of oversampling and separate processing of odd and even echo data. A Hamming filter was applied across all spatial dimensions to reduce peripheral lipid contamination.

This filter effectively increased the voxel volume by approximately 50%. Automatic frequency shift and zero-order phase correction based on the (residual) water signal was applied on a voxel-by-voxel basis. Odd and even spectra were summed to obtain NWS and WS spectral arrays. Reconstructed spectral quality was examined on the scanner in the Spectroscopy Task Card. Spectral postprocessing with LCModel fitting to generate metabolic maps was performed. Spectroscopic data from the top-most slice, which is partly covered by the OVS slices at the vertex of the head, were not analyzed.

### Quantification of Volume Coverage in Peripheral Lipid Containing Regions and in Lateral Gray Matter Regions

The MPRAGE scans of 11 of 14 patients were segmented using FreeSurfer (FS) segmentation pipeline (version 3.0.4) with optimized parameters using 50-100 mm smoothing distance (see results) and used as ground truth after converting to volumetric data. Regions outside of the outer GM surface were considered peripheral lipid containing regions and CSF. The percentage coverage of peripheral lipid containing regions and CSF was computed as the fraction of MRI voxels in this region that intersects with any of the OVS slices. Due to the finite transition bandwidth and the planar geometry of the OVS slices there is unavoidable suppression of lateral gray matter (GM) and white matter (WM) regions when using a finite number of OVS slices. The percentage brain tissue (GM+WM) loss was defined as the fraction of MRI voxels in the combined GM and WM masks, within the MRSI slab, that intersects with any of the OVS slices.

### Quantification of Residual Peripheral Lipid Signals

A peripheral mask was calculated based on the NWS and the WS images. Both NWS and WS were integrated along the spectral domain in magnitude mode and thresholded at 10% signal intensity to create masks that defined the inner volume of interest and the entire imaged slab. Subtracting these two masks created the peripheral mask. For five central slices within the selected MRSI slab the residual integrated lipid signal for all voxels in the peripheral mask was computed by integrating the area under the main lipid peak (1.3 ppm) in the water suppressed data in magnitude mode over the range 1.02 ppm-1.67 ppm.

### Parameter Settings for Image Registration

The factor that affected the reliability of registration between atlas space and subject space most was the image intensity inhomogeneity. Therefore, the intensity inhomogeneities was corrected using the N3 method with a relatively small smoothing distance of 50-100 mm. Based on the intensity-corrected image, the correlation ratio as a similarity measure for multimodal image registration was the best cost function for the data, and it resulted in the smallest position variability of the slice prescriptions (center and normal vector).

### Offline Validation of Automatic Placement of Up to 16 OVS Slices

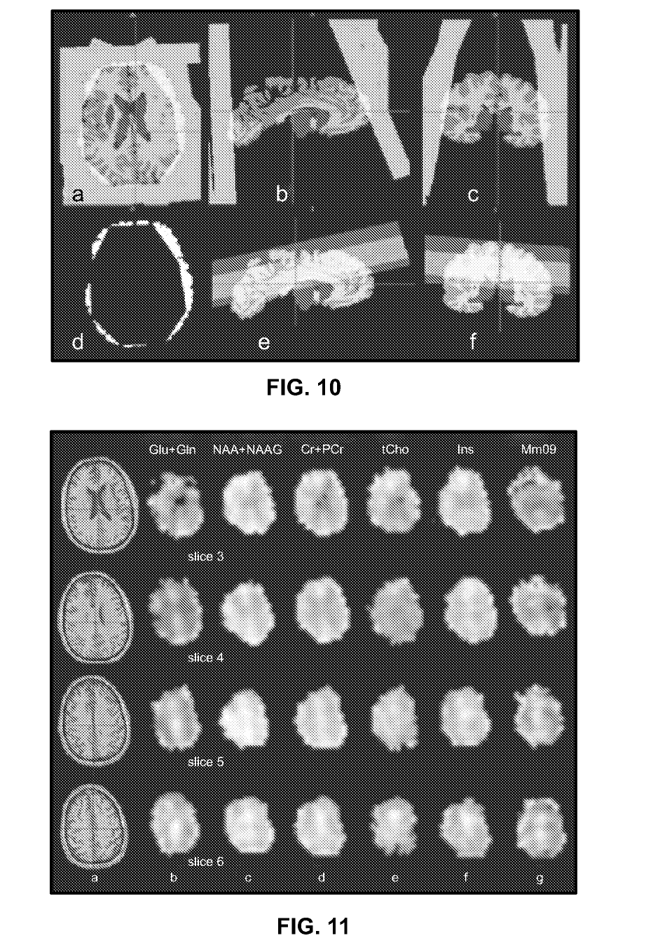

When transforming the MRSI slab from MNI space (FIG. 8(a)) to subject space (FIG. 8(c)), the MRSI slab was oriented and placed at an equivalent location extending from the middle of the ventricles in superior direction, consistent with the targeted VOI for 3D MRSI. The positioning of the OVS slices in subject space (FIG. 8(d)) was also consistent with the placement in MNI space (FIG. 8(b)), forming a convex hull around the upper cerebrum. In both atlas and subject space the peripheral lipid containing regions were 100% covered by the automatically placed OVS slices in all 11 subjects, both for 8 and for 16 OVS slices (FIG. 9). Placement of MRSI slab and 16 OVS slices on the MNI template head and transformation to the subject head, displayed in the mid-sagittal plane, in offline computation. The MRSI slab and OVS slices 1 and 16 are depicted in the MNI head FIG. 9(a) and mapped onto the subject's head FIG. 9(c). OVS slices 2 through 15 are displayed on the MNI head FIG. 9(b) and mapped onto the subject's head FIG. 9(d).

The brain tissue loss in the 40 mm MRSI slab using 8 OVS slices was 13.9% on average for the 11 subjects (Table 4).

TABLE 4
Offline computation of brain tissue loss in lateral gray
matter due to automated placement of 16 versus 8 OVS slices
and increase in brain volume coverage for 16 versus 8 OVS
slices using MPRAGE data collected in 11 subjects.

| | Brain tissue loss | Brain tissue loss | Increase in brain |
|---|---|---|---|
| | for 16 OVS | for 8 OVS | volume coverage— |
| Subject | slices [%] | slices [%] | 16 vs. 8 OVS slices |
| 1 | 9.79 | 10.74 | 1.6069 |
| 2 | 22.07 | 12.79 | 1.5979 |
| 3 | 22.71 | 13.42 | 1.6032 |
| 4 | 16.22 | 9.68 | 1.5816 |
| 5 | 22.39 | 14.67 | 1.5651 |
| 6 | 22.75 | 13.60 | 1.5882 |
| 7 | 16.67 | 9.46 | 1.6105 |
| 8 | 18.04 | 11.27 | 1.5958 |
| 9 | 19.12 | 17.88 | 1.6085 |
| 10 | 23.32 | 21.31 | 1.5772 |
| 11 | 18.78 | 17.89 | 1.5767 |
| Mean (SD) | 19.26 (4.07) | 13.88 (3.79) | 1.59 (0.02) |

Brain tissue loss is computed with respect to the brain volume encompassed by the MRSI slab. For the 80 mm MRSI slab using 16 OVS slices the brain tissue loss was 19.3%. This difference is in part due to the use of 6 OVS slices in the superior ring for the 16 OVS slice case, which leads to a coarser coverage of peripheral regions. Furthermore, the thicker slab for the 16 OVS slice case covered more brain tissue close to the top of the head, which exhibits much stronger curvature than the brain region imaged for the 8 OVS slice case. This makes suppression more difficult and necessitates sacrificing more brain tissue closer to the top of the brain. An example of the OVS placement and the degree of suppression of lateral gray matter for the 8 OVS slice case is shown in FIG. 10.

### Online Validation Comparing Automatic and Manual Placement of 8 OVS Slices

Automated prescription of the MRSI slab and the OVS slices (steps 2-6 in the Method Implementation) on the external workstation took less than four minutes and was performed while a T₂ weighted scan for manual placement was acquired. As with the offline simulations, complete coverage of peripheral lipid containing regions with automated OVS slice placement was obtained in all subjects and verified on the scanner console prior to collecting MRSI data. Volume coverage, spectral line width and lipid contamination for manual and automated OVS placement were comparable, enabling computation of metabolite maps of Inc., Cr+PCr, Glu+Gln, NAA+NAAG, and macromolecular resonances at 0.9 and 2.0 ppm. The Cramer Rao Lower Bound (CRLB) thresholds were 20% for NAA+NAAG, Cr+PCr, 30% for Ins and Cho+PCho, and 50% for Glu+GIn and MM9 (macromolecules at 0.9 ppm). Metabolite ratio maps obtained with automated OVS placement show relatively uniform metabolite distributions with distinct GM/WM contrast in Cho, Cr and Glu+GIn maps, consistent with previous studies (FIG. 11). The slice averaged metabolite concentration ratios for the 4 central slices that are fully encompassed within the MRSI slab shown in Table 5 are comparable for automated and manual placement.

TABLE 5
Slice averaged metabolite concentration ratios with respect to
Cr + PCr (standard deviation) in metabolite maps measured with automatic and
manual placement of 8 OVS slices as a function of slice position within the
MRSI slab. They are also comparable across slices, as expected, and
consistent with previous results. The number of usable voxels in these maps
with the above-described CRLB thresholds (Table 6) was comparable across
slices and metabolites, but 16% (on average) smaller for automatic compared
to manual placement, due to the more conservative choice of the OVS slice
thickness for automated placement.

| | | | NAA + | | | |
|---|---|---|---|---|---|---|
| | Placement | Glu + Gln/Cr + | NAAG/Cr + | | | MM09/Cr + |
| Slice | Method | PCr | PCr | tCho/Cr + PCr | Ins/Cr + PCr | PCr |
| 3 | Automatic | 1.08 (0.53) | 1.17 (0.43) | 0.26 (0.11) | 1.08 (0.33) | 1.43 (0.65) |
| | Manual | 1.56 (0.52) | 1.37 (0.73) | 0.44 (0.12) | 1.02 (0.33) | 1.24 (0.45) |
| 4 | Automatic | 1.26 (0.68) | 1.29 (0.43) | 0.26 (0.10) | 1.05 (0.31) | 1.64 (0.70) |
| | Manual | 1.21 (0.57) | 1.25 (0.44) | 0.24 (0.09) | 1.03 (0.29) | 1.47 (0.53) |
| 5 | Automatic | 1.32 (0.37) | 1.14 (0.37) | 0.22 (0.08) | 0.92 (0.33) | 1.37 (0.56) |
| | Manual | 1.43 (0.64) | 1.24 (0.36) | 0.24 (0.08) | 0.96 (0.27) | 1.44 (0.51) |
| 6 | Automatic | 1.68 (0.66) | 1.45 (0.57) | 0.23 (0.10) | 0.90 (0.33) | 2.20 (0.92) |
| | Manual | 1.15 (0.38) | 1.26 (0.35) | 0.26 (0.08) | 0.97 (0.28) | 1.45 (0.38) |
| Mean (SD) | Automatic | 1.34 (0.25) | 1.26 (0.14) | 0.25 (0.02) | 0.99 (0.09) | 1.66 (0.38) |
| Mean (SD) | Manual | 1.34 (0.19) | 1.28 (0.06) | 0.29 (0.10) | 1.00 (0.03) | 1.40 (0.11) |

TABLE 6
Number of voxels above threshold in metabolite maps acquired with
automatic and manual placement of 8 OVS slices as a function of slice
position within the MRSI slab for 3D data set with 8 encoded slices.
Residual lipid signals in peripheral regions of the MRSI with
automatically placed OVS slices were also comparable to data obtained
with manually placed OVS slices (Table 7) and within the variability
of lipid suppression ratios measured across subjects.

| | Place- | | | | | | |
|---|---|---|---|---|---|---|---|
| | ment | Glu + | NAA + | Cr + | | | |
| Slice | Method | Gln | NAAG | PCr | tCho | Ins | MM09 |
| 3 | Auto- | 212 | 237 | 235 | 213 | 229 | 234 |
| | matic | | | | | | |
| | Manual | 265 | 266 | 284 | 248 | 271 | 292 |
| 4 | Auto- | 225 | 223 | 226 | 207 | 223 | 277 |
| | matic | | | | | | |
| | Manual | 252 | 259 | 249 | 227 | 248 | 252 |
| 5 | Auto- | 206 | 206 | 211 | 181 | 197 | 231 |
| | matic | | | | | | |
| | Manual | 232 | 229 | 225 | 203 | 218 | 257 |
| 6 | Auto- | 205 | 189 | 195 | 162 | 188 | 242 |
| | matic | | | | | | |
| | Manual | 284 | 269 | 281 | 259 | 284 | 242 |
| Mean | Auto- | 212.0 | 213.7 | 216.8 | 190.8 | 209.3 | 246.0 |
| (SD) | matic | (9.2) | (20.8) | (17.6) | (23.7) | (19.8) | (21.2) |
| Mean | Manual | 258.3 | 255.8 | 259.8 | 234.3 | 255.3 | 260.8 |
| (SD) | | (21.9) | (18.3) | (28.1) | (24.7) | (29.0) | (21.7) |

TABLE 7
Comparison of the integrated residual lipid signal for
automatic and manual placements for five slices of selected
3D PEPSI data sets with 8 encoded slices.

| | Automatic | Manual |
|---|---|---|
| Slice | Placement | Placement |
| 3 | 1.00 | 0.86 |
| 4 | 1.00 | 0.91 |
| 5 | 0.97 | 0.94 |
| 6 | 0.86 | 0.89 |
| 7 | 0.85 | 0.63 |
| Mean | 0.94 | 0.84 |
| (SD) | (0.07) | (0.13) |

The integrated lipid signal (arbitrary units) is scaled to the maximum in slices 3 and 4 of the data set acquired with automated placement.

### Online Validation Using Automated Placement of 16 OVS Slices

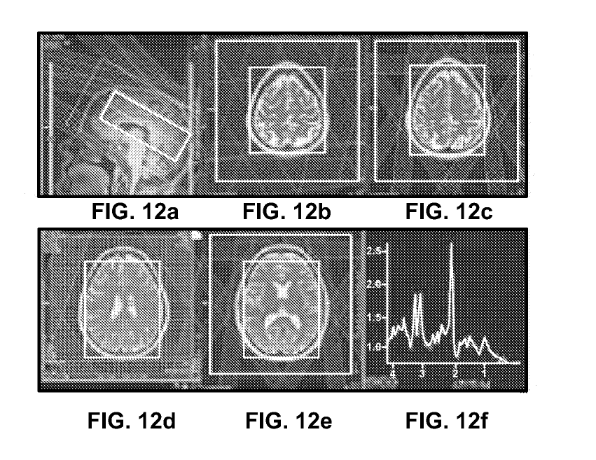

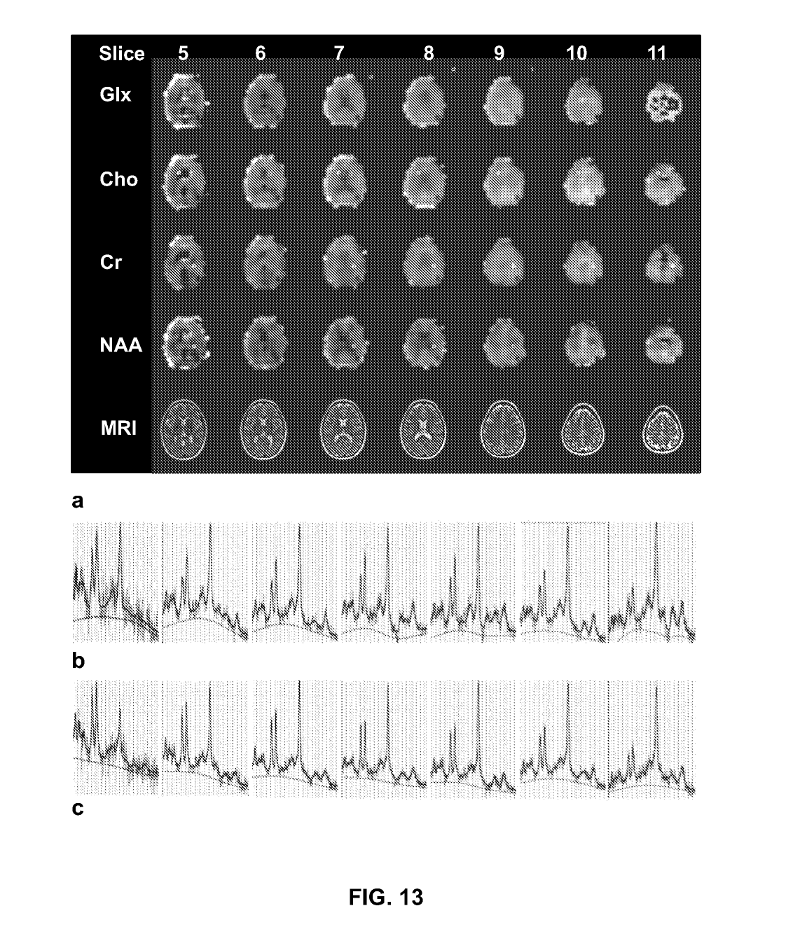

Complete coverage of lipid containing regions was obtained in all three subjects in which 16 OVS slices were used and verified on the scanner console prior to collecting MRSI data by assessing the placement of the OVS slices (FIG. 12). The spectral quality and degree of lipid suppression was comparable to the 8 OVS slice case (FIG. 12(f)). Metabolite maps shown in FIG. 13(a) display image quality comparable to the previous studies with respect to uniformity, but much larger volume coverage than previous short TE studies. The small voxel size and the stronger slab angulation in this study limited magnetic field inhomogeneity related line broadening and baseline distortion. Selected spectra from lateral and central regions in different slice locations demonstrate comparably low levels of lipid contamination (FIGS. 13(b) and 13(c)), confirmed by their respective LC model fits.

A method for positioning OVS slices and the MRSI slab based on an anatomical brain atlas is disclosed, which eliminates the need for any operator interaction for collecting MRSI data. This method uses atlas based registration for OVS in MRSI. This approach follows the use of statistical brain atlases in clinical settings for automatic positioning of MR imaging slices, which has become available as a product on clinical MRSI scanners. Landmark based methods have also gained popularity for positioning MRI slices in clinical imaging, but large number of landmarks would be required for placement of OVS slices due to the complex geometry of the surface of the brain, facial regions and peripheral lipid containing regions. Automatic placement of 8 OVS slices provides consistent short TE MRSI volume selection and comparable spectral quality across subjects with a similar degree of lipid suppression and only slightly reduced number of usable voxels as manual placement due to more conservative choice of the OVS slice thickness for automated placement. Short TE MRSI with 16 automatically placed OVS slices and MRSI slab is possible, and larger volume coverage may be achieved while maintaining similar spectral quality and degree of lipid suppression as in other methods. The number of OVS slices is limited by T₁ relaxation during the application of the OVS modules and by nonuniformity of the B₁ field. Repetitions of the OVS modules may be required to achieve adequate suppression. Additional OVS modules to augment lipid suppression in presaturated OVS slices or to define additional OVS slices may be inserted into a spin echo sequence and during the TM period of a stimulated echo pulse sequence.

The use of an atlas-based approach has two advantages compared to iterative optimization methods. First, the computational burden is considerably reduced facilitating integration of OVS placement into the scanner workflow. Second, iterative optimization methods may converge in local minima, resulting in suboptimal saturation band placement and possible inter-subject variation in saturation band placement. The atlas based approach is expected to increase consistency of OVS placement and MRSI slab selection between subjects and during scan repetitions, which is advantageous for longitudinal and cross-sectional studies. Iterative optimization on the other hand is suitable for offline generation of optimal OVS slice and MRSI slab positions in atlas space under the supervision of an experienced user, who ensures that the global optimum is selected. The automated OVS positioning method by Ryner et al. and Venugopal et al. is based on optimization in subject space, but has not yet been applied to delineate an entire organ. The automated prescription method by Ozhinsky and Nelson, which employs prelocalization using an oblique PRESS box and 9 OVS slices to extend brain coverage, also operates in subject space. These methods are capable of accurate placement of OVS slices on a patient by patient basis, but they are subject to brain segmentation errors and optimization reliability. Since they do not guarantee consistent OVS coverage and ROI positioning, these methods are not suitable for investigating spectral changes in lateral cortical regions in longitudinal studies in individual patients and in cross-sectional studies.

The sensitive volume in this study was limited by shimming considerations rather than by the number of OVS slices. Larger volume coverage at short TE would be possible, if shimming conditions in frontal and inferior temporal cortex were improved. Reducing voxel size to 0.37 cc in this study helped mitigate magnetic field inhomogeneity in frontal brain areas and reduced spatial contamination from peripheral lipid signals.

Consistent with previous implementations of OVS slices, a planar geometry is used for OVS slices. Although this approach provides satisfactory coverage as shown in the data, there may be cases where unusual skull shapes and large thickness of peripheral regions may lead to inadequate coverage of lipid containing regions. As a potential solution, nonlinear registration using spatial normalization could be used for more accurate transformation between the atlas space and the subject space. In the future, a non-planar geometry of OVS slice prescription from nonlinear transformation may be feasible using curved slice excitation, taking advantage of parallel transmit technology.

While the results of automated placement are generally comparable to those obtained with manual placement by an experienced operator, the usable volume of interest was slightly smaller than with manual placement due to conservative choice of OVS slice thickness in atlas space to accommodate inter-individual differences in local brain shapes and thickness of peripheral lipid containing regions. Patients vary in skull and peripheral tissue thickness; thus, an optimal affine transformation for the brain may not be optimal for the skull. Hence, in the current in vivo experiments, a fixed and conservative thickness (25 mm) of the OVS slices in MNI space is used, which proved adequate for all patients. Brain segmentation could be used online to positively identify peripheral regions to constrain the affine transformation, thus enabling thinner OVS slices to be positioned in patient space resulting in larger volume coverage.

The feasibility of automatic and optimal placement of OVS slices based on an atlas brain is demonstrated. The overall quality of metabolite maps obtained when OVS slices are automatically positioned matches that of maps with manually placed OVS slices. Atlas-based prescription of the MRSI slab ensures operator independent and pre-specified selection of volume of interest across subjects. It also provides the flexibility to rapidly place large numbers of OVS slices. Moreover, it has the potential to improve the reliability of clinical 3D MRSI at short TE to reduce operator bias and speed up clinical throughput. Atlas-based auto-placement prescription of MRSI slab and OVS slices is thus advantageous for longitudinal and cross sectional clinical MRSI studies and may be integrated with automated MRI slice prescription software on clinical MR scanners.

While the present invention and what is considered presently to be the best modes thereof have been described in a manner that establishes possession thereof by the inventors and that enables those of ordinary skill in the art to make and use the inventions, it will be understood and appreciated that there are many equivalents to the exemplary embodiments disclosed herein and that myriad modifications and variations may be made thereto without departing from the scope and spirit of the invention, which is to be limited not by the exemplary embodiments but by the appended claims.

What is claimed is:

1. A system for imaging a patient organ, comprising:
an imaging apparatus;
a memory configured to store code;
a processor communicating with said memory and imaging apparatus and configured to:
receive data corresponding to an alignment of the patient organ with a standardized organ;
spatially normalize the standardized organ to the patient organ;
provide optimized slices of the standardized organ;
translate the optimized slices of the standardized organ into optimized slices of the patient organ; and
provide instructions to said imaging apparatus for imaging the patient organ according to the optimized slices of the patient organ.

2. The system of claim 1, wherein the patient organ is a brain.

3. The system of claim 1, wherein the optimized slices comprises arbitrary number of outer volume suppression slices.

4. The system of claim 1, wherein the optimized slices comprises 16 outer volume suppression slices.

5. The system of claim 1, wherein the imaging apparatus is one of an MRI and MRSI imaging apparatus.

6. The system of claim 1, further comprising a display displaying scanned patient images.

7. A method for imaging a patient organ comprising the steps of:
aligning the patient organ with a standardized organ;
spatially normalizing the standardized organ to the patient organ;
providing optimized slices of the standardized organ;
translating the optimized slices of the standardized organ into optimized slices of the patient organ; and
imaging the patient organ according to the optimized slices of the patient organ.

8. The method of claim 7, wherein the standardized organ is an organ atlas.

9. The method of claim 7, wherein the standardized organ and the patient organ is a brain.

10. The method of claim 7, wherein the optimized slices is an arbitrary number of slices.

11. The method of claim 7, wherein the number of optimized slices is 16.

12. A method for imaging a patient organ comprising the steps of:
optimizing placement of one or more suppression slices in peripheral regions in reference to a standardized organ space;
automatically aligning a data acquisition coordinate system of an imaging apparatus so that an image of the patient organ is co-registered to the standardized organ space;
spatially normalizing the standard organ space into a patient organ space; and
transforming spatial coordinates and angles of suppression regions from the standardized organ space into the patient organ space using the spatial transformations of said automatically aligning step and said spatially normalizing step.

13. The method for imaging a patient organ of claim 12, further comprising a step of optimizing the suppression of peripheral regions around the patient organ.

14. The method of claim 13, wherein the selection of the suppression placement occurs in reference to segmented high-resolution magnetic resonance imaging.

15. The method of claim 13, wherein the selection of suppression placement occurs in reference to chemical-shift selective magnetic resonance imaging.

16. The method of claim 12, wherein the step of optimizing placement of one or more suppression slices is an automated step.

17. The method of claim 12, wherein the patient' organ is a brain.

18. The method of claim 12, wherein the spatial normalization step includes using an inverse of spatial normalization that transforms a patient space into a standardized space.

19. The method of claim 18, wherein spatial resolution is reduced.
